(19) United States
(12) Patent Application Publication
(10) Pub. No.: US 2024/0317700 A1
(43) Pub. Date: Sep. 26, 2024

(54) PROCESS FOR PREPARING GOSSYPETIN

(71) Applicant: NovMetaPharma Co., Ltd., Seoul (KR)

(72) Inventors: Soo Jin LEE, Cheongju-si (KR); Min Kyu KIM, Ansan-si, Gyeonggi-do (KR); Gyun Ho SON, Suwon-si, Gyeonggi-do (KR); Hoe Yune JUNG, Pohang-si, Gyeongsangbuk-do (KR); Heonjong LEE, Incheon (KR)

(73) Assignee: NovMetaPharma Co., Ltd., Seoul (KR)

(21) Appl. No.: 18/577,804

(22) PCT Filed: Jul. 15, 2022
(86) PCT No.: PCT/KR2022/010372

§ 371 Date: Jan. 9, 2024

(30) Foreign Application Priority Data
Jul. 16, 2021 (KR) 10-2021-0093258

(51) Int. Cl. C07D 311/30; C07H 17/07

(52) U.S. Cl. CPC C07D 311/30; C07H 17/07

(57) ABSTRACT

A method for efficiently preparing gossypetin is disclosed. Gossypetin can be utilized as an ingredient in medicine, healthcare, cosmetics, health foods, dietary supplements, and the like. The method can produce gossypetin of formula (1) with high purity and high yield through a four-step process, in which the compound of formula (2) is employed as starting material.

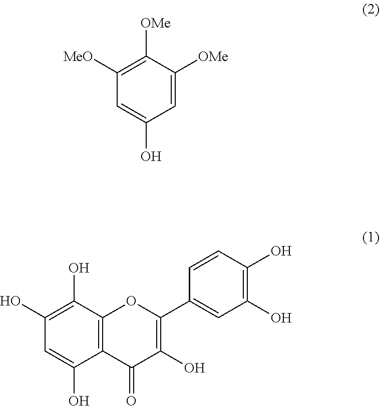 [SMILES: COc1cc(O)cc(OC)c1OC ; O=c1c(O)c(-c2ccc(O)c(O)c2)oc2c(O)c(O)cc(O)c12]

### TECHNICAL FIELD

The present invention relates to a process for preparing gossypetin. More particularly, the present invention relates to an efficient process for preparing gossypetin with high purity and high yield.

### BACKGROUND ART

Gossypetin is a type of flavonoid, a compound represented by the following formula (1).

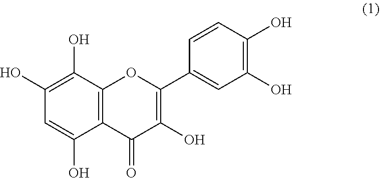 [SMILES: O=c1c(O)c(-c2ccc(O)c(O)c2)oc2c(O)c(O)cc(O)c12]

Gossypetin is isolated from the flowers and sepals of roselle (Hibiscus sabdariffa) and is known to exhibit strong antibacterial activity. In recent years, gossypetin has been reported to be effective in improving osteoporosis and preventing or treating neurodegenerative diseases, and has gained increasing interests as an ingredient in health functional foods as well as medicines [see Korean Patent Publication No. 2017-0124242 and Patent Registration No. 210288]. Furthermore, as an antioxidant, gossypetin can be utilized as an ingredient in medicine, healthcare, cosmetics, and health foods.

However, gossypetin is mainly extracted from natural products or synthesized through a complex process, making mass production difficult. Accordingly, there has been an urgent need to develop a method for industrial production of gossypetin.

### DISCLOSURE

### Technical Problem

It is an object of the present invention to provide an efficient process for preparing gossypetin with high purity and high yield.

### Technical Solution

One embodiment of the present invention relates to a process for preparing gossypetin of the following formula (1), which comprises the steps of:
(i) subjecting a compound of the following formula (2) to methylation to obtain a compound of the following formula (3);
(ii) subjecting the compound of the following formula (3) to Friedel-Crafts reaction to obtain a compound of the following formula (4);
(iii) reacting the compound of the following formula (4) with a compound of the following formula (5) in the presence of a base to obtain a compound of the following formula (6); and
(iv) subjecting the compound of the following formula (6) to demethylation.

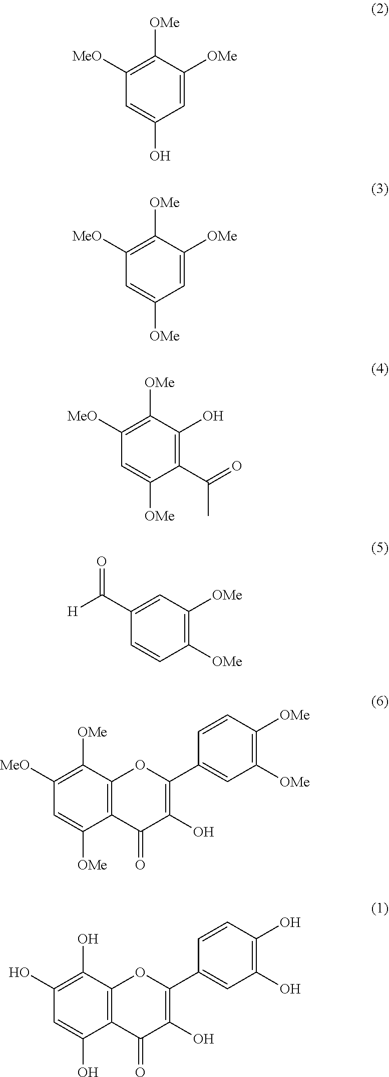

Hereinafter, the preparation process of the present invention is described in more detail referring to the following reaction scheme 1. The process depicted in the following reaction scheme 1 represents merely a typical example, and various changes may be made to reagents and reaction conditions without limitation.

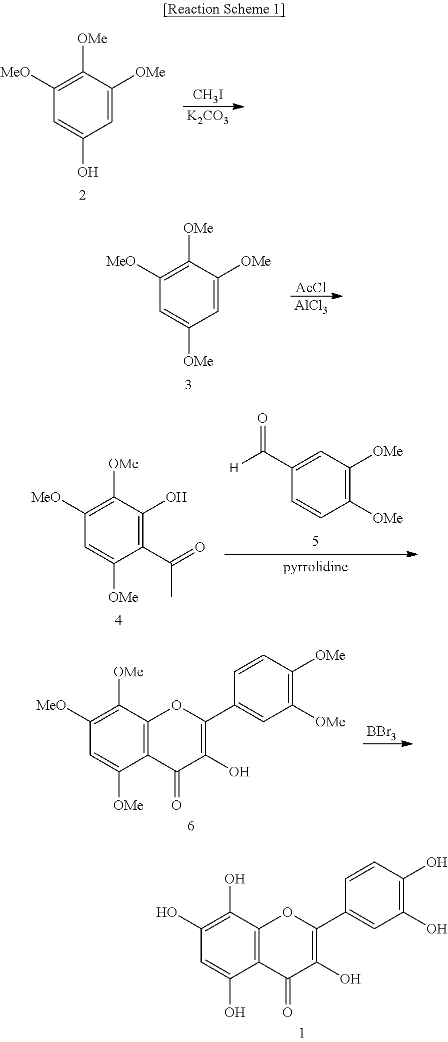

### Step 1: Synthesis of Compound of Formula (3)

The compound of formula (3) can be prepared by subjecting the compound of formula (2) to methylation.

The methylation may be carried out by reacting the compound of formula (2) with methyl halide, for example iodomethane, in the presence of a base.

As the base, potassium carbonate, cesium carbonate, etc. may be used. Particularly, potassium carbonate is preferred.

The reaction temperature is preferably about 50 to 70° C.

As the reaction solvent, acetone, N,N-dimethylformamide, etc. may be used. Particularly, acetone is preferred.

### Step 2: Synthesis of Compound of Formula (4)

The compound of formula (4) can be prepared by subjecting the compound of formula (3) to Friedel-Crafts reaction.

The Friedel-Crafts reaction can be carried out by reacting the compound of formula (3) with an acetyl halide in the presence of a Lewis acid.

As the Lewis acid, aluminum chloride, aluminum bromide, etc. may be used. Particularly, aluminum chloride is preferred.

As the acetyl halide, acetyl chloride, acetyl bromide, etc. may be used. Particularly, acetyl chloride is preferred.

The reaction temperature is preferably room temperature. As the reaction solvent, dichloromethane, diethyl ether, etc. may be used.

### Step 3: Synthesis of Compound of Formula (6)

The compound of formula (6) can be prepared by reacting the compound of formula (4) with the compound of formula (5) in the presence of a base.

As the base, a cyclic secondary amine such as pyrrolidine and piperidine may be used. Particularly, pyrrolidine is preferred.

The base may be used in an amount of 5 to 15 equivalents, preferably 8 to 12 equivalents, to 1 equivalent of the compound of formula (4).

The reaction temperature is preferably about 20 to 80° C., more preferably 40 to 60° C.

The reaction solvent is preferably a protic solvent such as water, methanol, ethanol, and especially water.

The reaction may be carried out in air or under an oxygen atmosphere.

According to one embodiment of the present invention, the compound of formula (4) and the compound of formula (5) are subjected to aldol condensation followed by cyclization and oxidation, simultaneously, as shown in the following reaction scheme 2, such that the compound of formula (6) can be prepared in a one-step reaction.

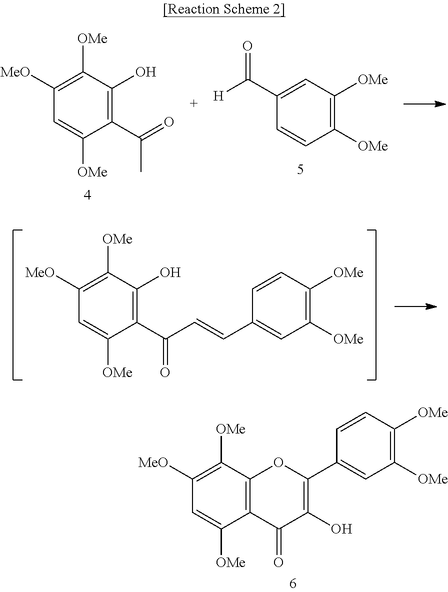

This increases chemical selectivity and prevents the formation of isomers, resulting in higher yield and easier purification.

### Step 4: Preparation of Gossypetin of Formula (1)

The compound of formula (1) can be prepared by subjecting the compound of formula (6) to demethylation.

The demethylation can be carried out in the presence of a catalyst such as boron tribromide and aluminum tribromide.

The reaction temperature is preferably room temperature, and dichloromethane, acetonitrile, etc. may be used as the reaction solvent.

### Advantageous Effects

In accordance with the preparation process of the present invention, gossypetin with high purity can be efficiently prepared in a four-step process with high yields.

### BEST MODE

The present invention will be described in more detail by following examples. It will be obvious to those skilled in the art that these examples are merely described for illustration of the present invention and the scope of the present invention is not limited thereto.

### Example 1: Synthesis of Compound of Formula (3)

The compound of formula (2) (500 g) was dissolved in acetone (3,000 mL), and anhydrous potassium carbonate (375.18 g) was added. Then, CH₃I (500.90 g) was added, and the reaction mixture was stirred at 60° C. overnight. The reaction solution was concentrated, and the crude product was subjected to silica gel column chromatography to obtain the title compound (470 g, 87% yield).

¹H NMR (400 MHZ, CDCl₃): 6.15 (2H), 3.85 (6H), 3.78 (6H)

### Example 2: Synthesis of Compound of Formula (4)

The compound of formula (3) (470 g) was dissolved in dichloromethane, and AlCl₃ (474.21 g) and acetyl chloride (241.97 g) were added. The reaction mixture was stirred at room temperature overnight, and the crude product was subjected to silica gel column chromatography to obtain the title compound (440 g, 82% yield).

¹H NMR (400 MHz, CDCl₃): 13.80 (1H), 5.98 (1H), 3.94 (3H), 3.89 (3H), 3.81 (3H), 2.62 (3H)

### Example 3: Synthesis of Compound of Formula (6)

The compound of formula (4) (440 g) and the compound of formula (5) (356 g) were dissolved in water (1,800 g), pyrrolidine (152.13 g) was added, and the reaction mixture was stirred at 50° C. in air overnight. The crude product was subjected to silica gel column chromatography to obtain the title compound (117 g, 30% yield).

¹H NMR (400 MHZ, CDCl₃): 7.93 (t, 2H), 7.41 (s, b, 1H), 7.05 (d, 1H), 6.44 (1H), 4.04-3.99 (m, 15H)

¹³C NMR (100 MHz, CDCl₃): 172.26, 156.67, 156.16, 150.92, 150.36, 148.87, 142.18, 137.30, 130.54, 124.02, 124.02, 120.90, 111.08, 110.15, 106.07, 91.75, 61.50, 56.52, 56.47, 56.02, 55.89

### Example 4: Synthesis of Compound of Formula (1)

The compound of formula (6) (117 g) was dissolved in dichloromethane, and BBr₃ (1,132 g) was added at −78° C. The reaction mixture was stirred at room temperature overnight. The crude product was subjected to silica gel column chromatography to obtain the title compound (80 g, 83.4% yield).

Then, prep-HPLC (mobile phase: A: water (0.2% TFA), B: acetonitrile (0.2% TFA); Gradient: 5%-45%, 25 min) was carried out to obtain the title compound with 99.45% purity (32.5 g, single impurity less than 0.5%).

¹H NMR (400 MHZ, DMSO-d⁶): 11.92 (1H), 10.41 (1H), 9.57 (1H), 9.28 (2H), 8.60 (1H), 7.79 (1H), 7.67 (1H), 6.90 (1H), 6.27 (1H)

¹³C NMR (100 MHZ, DMSO-d⁶): 176.56, 153.11, 152.67, 148.14, 147.11, 145.46, 145.35, 136.92, 125.18, 122.75, 120.66, 115.94, 115.75, 103.21, 98.42

1. A process for preparing gossypetin of the following formula (1), which comprises the steps of:
(i) subjecting a compound of the following formula (2) to methylation to obtain a compound of the following formula (3);
(ii) subjecting the compound of the following formula (3) to Friedel-Crafts reaction to obtain a compound of the following formula (4);
(iii) reacting the compound of the following formula (4) with a compound of the following formula (5) in the presence of a base to obtain a compound of the following formula (6); and
(iv) subjecting the compound of the following formula (6) to demethylation:

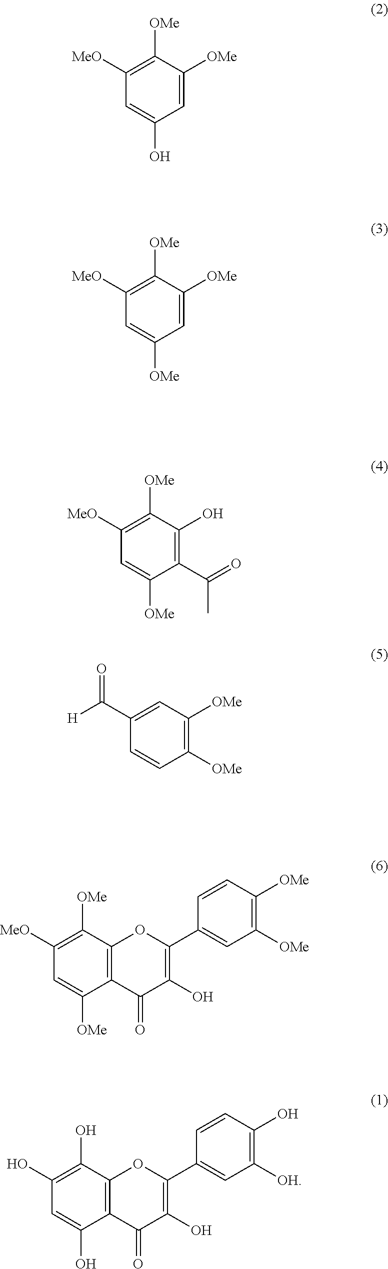 [SMILES: COc1cc(O)cc(OC)c1OC ; COc1cc(OC)c(OC)c(OC)c1 ; COc1cc(OC)c(C(C)=O)c(O)c1OC ; COc1ccc(C=O)cc1OC ; COc1ccc(-c2oc3c(OC)c(OC)cc(OC)c3c(=O)c2O)cc1OC ; O=c1c(O)c(-c2ccc(O)c(O)c2)oc2c(O)c(O)cc(O)c12]

2. The process according to claim 1, wherein the methylation of step (i) is carried out by reacting the compound of formula (2) with methyl halide in the presence of a base.

3. The process according to claim 1, wherein the Friedel-Crafts reaction of step (ii) is carried out by reacting the compound of formula (3) with an acetyl halide in the presence of a Lewis acid.

4. The process according to claim 1, wherein the base of step (iii) is a cyclic secondary amine.

5. The process according to claim 1, wherein the base of step (iii) is pyrrolidine.

6. The process according to claim 1, wherein the base of step (iii) is used in an amount of 8 to 12 equivalents to 1 equivalent of the compound of formula (4).

7. The process according to claim 1, wherein the reaction temperature of step (iii) is 40 to 60° C.

8. The process according to claim 1, wherein the reaction solvent of step (iii) is a protic solvent.

9. The process according to claim 1, wherein the reaction solvent of step (iii) is water.

10. The process according to claim 1, wherein the reaction of step (iii) is carried out in air.

11. The process according to claim 1, wherein the demethylation of step (iv) is carried out in the presence of boron tribromide.
